(19) United States
(12) Patent Application Publication
(10) Pub. No.: US 2022/0088269 A1
(43) Pub. Date: Mar. 24, 2022

(54) METHOD OF DEHYDRATION OF EXTRACELLULAR MATRIX AND PARTICLES FORMED THEREFROM

(71) Applicant: Miromatrix Medical Inc., Eden Prairie, MN (US)

(72) Inventors: Ben Steiner, Bloomington, MN (US); Dominique Davidow, Minnetonka, MN (US); Aleksandr Katane, Chaska, MN (US); Jeffrey Ross, Chaska, MN (US)

(21) Appl. No.: 17/420,099

(22) PCT Filed: Jan. 7, 2020
(86) PCT No.: PCT/US2020/012587

§ 371 Date: Jun. 30, 2021

Related U.S. Application Data
(60) Provisional application No. 62/789,218, filed on Jan. 7, 2019.

(51) Int. Cl. A61L 27/36; A61L 27/52; A61L 27/22; B33Y 80/00

(52) U.S. Cl. CPC A61L 27/3633; A61L 27/52; B33Y 70/00; B33Y 80/00; A61L 27/3691; A61L 27/22

(57) ABSTRACT

A method of extracellular matrix (ECM) particle formation, and compositions produced by the method, are provided.

### CROSS-REFERENCE TO RELATED APPLICATIONS

This application claims the benefit of the filing date of U.S. application No. 62/789,218, filed on Jan. 7, 2019, the disclosure of which is incorporated by reference herein.

### BACKGROUND

Three-dimensional (3D) printing is becoming an increasingly common technique to fabricate scaffolds and devices for tissue engineering applications. This is due to the potential of 3D printing to provide patient-specific designs, high structural complexity, and rapid on-demand fabrication at a low-cost. One of the major bottlenecks that limits the widespread acceptance of 3D printing in biomanufacturing is the lack of diversity in “biomaterial inks”.

Many printable materials have great properties for external applications, but implantable biomaterials require specific characteristics based on both physiological conditions and interactions with the body that make development much more difficult. In general, printable biomaterials must: (1) be printable, (2) be biocompatible, (3) have appropriate mechanical properties, (4) have good degradation kinetics, (5) form safe degradation byproducts, and (6) exhibit tissue biomimicry. How to fulfill each of these requirements varies slightly depending on which printing method is being used and the projected end application of the device. Furthermore, many of these characteristics can work against each other. For example, in bone tissue, it is desirable to have stiff materials for osteoblast development and load bearing, however, this can lead to either slow to nonexistent degradation. Soft materials can be printable find quicker to biodegrade, however, their ability to be handled and applied to certain tissue types may be a concern. The majority of 3D printed constructs are used in bone or cartilage applications due to the inherent stiffness of most printed biomaterials mimicking the natural stiffness of these tissues, outside of some hydrogel systems.

### SUMMARY

The disclosure provides a method to dehydrate decellularized extracellular matrix from, a mammalian organ, e.g., a pig or human organ such as liver, pancreas, kidney, lung, spleen, or heart, including portions thereof having dimensions, for instance, of about 10×Y inches, 5×Y inches, 2×Y inches, 1×Y inches, 0.5×Y inches, 0.25× Y inches, or 0.1×Y inches, where Y may be from 0.1 to 1 inches, 0.5 to 5 inches, 0.2 to 1 inches, 1 to 5 inches or 1 to 10 inches, in the absence of applied heat, e.g., the dehydration occurs at temperatures less than about 75° F., less than about 70° F., less than about 68° F., less than about 25° C., less than about 22° C. or less than about 19° C. In one embodiment, the portions may be compressed before hydration. For example, the thickness of a portion of the decellularized extracellular matrix from a mammalian organ may be compressed by at least 0.01%, 0.5%, 1%, 5%, 10%, 20%), 50%, 90% or more. In one embodiment, the dehydrated portion retains as much of the native organ composition as possible after ambient temperature dehydration, and optional compression, which removes moisture from, the decellularized organ, portion. The dehydrated materials are then subjected to milling, e.g., cryomilling, to provide for a population of particles ranging in size from about 0.001 to 0.005 mm up to about 10 mm, including about 0.01 mm to about 0.05 mm, about 0.05 mm to about 0.1 mm, 0.1 mm to about 5 mm, about 0.25 mm to about 0.5 mm, about 0.4 mm to about 4 mm, about 1 mm to about 2 mm, about 0.5 to about 1.5 mm, about 1.5 mm to about 3 mm, or about 3 mm to about 4 mm, which range may be obtained via milling or milling and sizing, e.g., using a sieve or other size separation device or method. The native composition of the extracellular matrix of whole organs provides the structure in the dehydrated and milled extracellular matrix particles to enable therapies or to prepare an organ specific ink to utilize in the printing of 3D structures to assist with organ function. Inks may be prepared by sizing the dehydrated particles prior to combining in a solvent, for instance, an aqueous solvent such as water or phosphate buffered saline (PBS) or by subjecting the dehydrated particles to chemical or enzymatic digestion. Bioprinting of tire inks may be via any method including but not limited to thermal inkjet bioprinting, piezoelectric inkjet bioprinting, pneumatic extrusion bioprinting, mechanical extrusion bioprinting or laser-assisted bioprinting. Thus, the particles may be employed in compositions useful in methods including but are not limited to ex vivo cellular assays, or the preparation of tissue engineered constructs for in vivo applications, e.g., printed structures based on perfusion decellularized ECM that are implanted for in vivo remodeling or combined with cells to provide a functional implant.

### BRIEF DESCRIPTION OF FIGURES

FIG. 1. Moisture content analysis.

FIG. 2. Particle size analysis.

FIG. 3. Gel showing sizes of product of perfusion decellularized liver extracellular matrix (dehydrated ECM; dECM) dried in a non-destructive method (e.g., at ambient temperature). 1) MW marker (Biorad); 2) 1 mg/mL, 10 μL; 3) 1 mg/mL, 10 μL; 4) 2.5 mg/mL; 5) 2.5 mg/mL; 6) 5 mg/mL; 7) 5 mg/mL; 8) 10 mg/mL; 9) 10 mg/mL; 10) 1 mg/mL, 10 μL; 11) 1 mg/mL, 10 μL; 12) 2.5 mg/mL; 13) 2.5 mg/mL; 14) 5 mg/mL; 15) 5 mg/mL. Lanes 2-9, no NaOH; lanes 10-15, NaOH added. Even numbered lanes have samples with reactions conducted at room temperature and odd numbered lanes have samples with reactions conducted at 4° C.

FIG. 4. Examples of drying perfusion decellularized matrix and cryomilling.

FIG. 5. Example of dECM-liver particle preparation and storage.

FIG. 6. Moisture content analysis.

### DETAILED DESCRIPTION

The disclosure provides for a decellularized particle product prepared by the dehydration of decellularized whole organ material, e.g., perfusion decellularized whole organ material. Many dehydration methods involve elevated temperatures to achieve liquid removal. The present method involves selecting or sizing of decellularized material, optional compression, followed by spacing in, for example, a laminar flow hood, for a defined amount of time. In one embodiment, the dehydration results in a moisture content in the product that is less than 10%, e.g., less than about 9%, 8%, 7%, or 5%. In one embodiment, compression and dehydration results in a moisture content in the product that is less than 8%, e.g., less than about 7%, 6%, 5%, or 4%. The ambient temperature of the dehydration process is non-destructive for any extracellular matrix components, resulting in a product that likely has higher efficacy. The material can then be cryomilled into a fine particle powder which can be used in a number of embodiments including but not limited to: 3D printing as a fine particle or digested into a solution that can be printed via reorganization by temperature, chemical cross-linking, UV cross-linking or other means, dissolving into a liquid for gel formation, including but not limited to protease digestion, dissolved into a liquid for use as an ink of 3D printing. The particles can also have direct therapeutic potential such as the direct application to wounds, filling of voids, intramuscular injections, application to tunneling wounds and fistulas.

### Sources of Decellularized ECM

In one embodiment, a method to decellularize a mammalian organ or tissue includes cannulating the organ or tissue. The vessels, ducts, and/or cavities of an organ or tissue can be cannulated using methods find materials known in the art. The next step in decellularizing an organ or tissue is to perfuse the cannulated organ or tissue with a cellular disruption medium. Perfusion through an organ can be multi-directional (e.g., antegrade and retrograde).

Langendorff perfusion of a heart is routine in the art, as is physiological perfusion (also known as four chamber working mode perfusion). See, for example, Dehnert, The Isolated Perfused Warm-Blooded Heart According to Langendorff, In Methods in Experimental Physiology and Pharmacology: Biological Measurement Techniques V. Biomesstechnik-Verlag March GmbH, West Germany, 1988. Briefly, for Langendorff perfusion, the aorta is cannulated and attached to a reservoir containing cellular disruption medium. A cellular disruption medium can be delivered in a retrograde direction down the aorta either at a constant flow rate delivered, for example, by an infusion or roller pump or by a constant hydrostatic pressure. In both instances, the aortic valves are forced shut and the perfusion fluid is directed into the coronary ostia (thereby perfusing the entire ventricular mass of the heart), which then drains into the right atrium via the coronary sinus. For working mode perfusion, a second cannula is connected to the left atrium and perfusion can be changed from retrograde to antegrade. Methods are known in the art for perfusing other organ or tissues including long, liver, pancreas, spleen, kidney, brain, and limbs.

One or more cellular disruption media can be used to decellularize an organ or tissue. A cellular disruption medium, generally includes at least one detergent such as SDS, PEG, or Triton X. A cellular disruption medium can include water such that the medium is osmotically incompatible with the cells. Alternatively, a cellular disruption medium can include a buffer (e.g., PBS) for osmotic compatibility with the cells. Cellular disruption media also can include enzymes such as, without limitation, one or more collagenases, one or more dispases, one or more DNases, or a protease such as trypsin. In some instances, cellular disruption media also or alternatively can include inhibitors of one or more enzymes (e.g., protease inhibitors, nuclease inhibitors, and/or collagenase inhibitors).

In certain embodiments, a cannulated organ or tissue can be perfused sequentially with two different cellular disruption media. For example, the first cellular disruption medium can include an anionic detergent such as SDS and the second cellular disruption medium can include an ionic detergent such as Triton X-100. Following perfusion with at least one cellular disruption medium, a cannulated organ or tissue can be perfused, for example, with wash solutions and/or solutions containing one or more enzymes such as those disclosed herein.

Alternating the direction of perfusion (e.g., antegrade and retrograde) can help to effectively decellularize the entire organ or tissue. Decellularization as described herein essentially decellularizes the organ from the inside out, resulting in very little damage to the ECM. An organ or tissue can be decellularized at a suitable temperature between 4 and 40° C. Depending upon the size and weight of an organ or tissue and the particular detergent(s) and concentration of detergent(s) in the cellular disruption medium, an organ or tissue generally is perfused from about 2 to about 12 hours per gram of solid organ or tissue with cellular disruption medium. Including washes, an organ may be perfused for up to about 12 to about 72 hours per gram of tissue. Perfusion generally is adjusted to physiologic conditions including pulsatile flow, rate and pressure.

A decellularized organ or tissue consists essentially of the extracellular matrix (ECM) component of all or most regions of the organ or tissue, including ECM components of the vascular tree. ECM components can include any or ail of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue.

The morphology and architecture of the ECM (prior to dehydration) can be examined visually and/or histologically. For example, the basal lamina on the exterior surface of a solid organ or within the vasculature of an organ or tissue should not be removed or significantly damaged due to decellularization. In addition, the fibrils of the ECM should be similar to or significantly unchanged from that of an organ or tissue that has not been decellularized. Unless indicated otherwise, decellularization as used herein refers to perfusion decellularization and, unless indicated otherwise, a decellularized organ or matrix referred to herein is obtained using the perfusion decellularization described herein. Perfusion decellularization as described herein can be compared to immersion decellularization as described, for example, in U.S. Pat. Nos. 6,753,181 and 6,376,244. Either method can be used as a source of ECM for the methods disclosed herein.

Thus, decellularization of a solid organ removes most or all of the cellular components while substantially preserving the extracellular matrix (ECM) and the vasculature bed. Mammals from which solid organs can be obtained include, without limitation, rodents, pigs, rabbits, cattle, sheep, dogs, and humans. Organs and tissues used in the methods described herein can be cadaveric, or can be fetal, neonatal, or adult. Solid organs as referred to herein include, without limitation, heart, liver, lungs, skeletal muscles, brain, pancreas, spleen, kidneys, stomach, uterus, and bladder. In one embodiment, a solid organ refers to an organ that has a “substantially closed” vasculature system. A “substantially closed” vasculature system with respect to an organ means that, upon perfusion with a liquid, the majority of the liquid is contained within the solid organ and does not leak out of the solid organ, assuming the major vessels are cannulated, ligated, or otherwise restricted. Despite having a “substantially closed” vasculature system, many of the solid organs listed above have defined “entrance” and “exit” vessels which are useful for introducing and moving the liquid throughout the organ during perfusion.

In addition to the solid organs described above, other types of vascularized organs or tissues such as, for example, all or portions of joints (e.g., knees, shoulders, hips or vertebrae), trachea, skin, mesentery or gut, small and large bowel, esophagus, ovaries, penis, testes, spinal cord, or single or branched vessels can be decellularized using the methods disclosed herein. Further, the methods disclosed herein also can be used to decellularize avascular (or relatively avascular) tissues such as, for example, cartilage or cornea.

A decellularized organ or tissue as described herein (e.g., heart or liver) or any portion thereof (e.g., an aortic valve, a mitral valve, a pulmonary valve, a tricuspid valve, a pulmonary vein, a pulmonary artery, coronary vasculature, septum, a right atrium, a left atrium, a right ventricle, a left ventricle or a hepatic lobe), may be employed a source of ECM for the method to dehydrate and to form ECM based particles.

### Exemplary Method

Prior to dehydration, decellularized material may be processed in several batches and stored for the purposes of pooling and processing together at a later date. In one embodiment, the method involves incubation of compressed decellularized whole organ material in a pH neutral disinfectant, e.g., 500 ppm peracetic acid (PAA), bath (5-10 L) for about 5-minutes with gentle agitation. Other disinfectants may be employed including but not limited to lactic acid, alcohol, hydrogen peroxide, hypochlorite, dioxide, sodium dichloroisocyanurate, chloramine-T and the like. In one embodiment, this is followed by a 5-minute deionized water bath (5-10 L) with gentle agitation. After the disinfection and wash, material may be aseptically packaged with deionized water or other physiologically compatible solutions to keep material from drying out. Packaging is then stored at 4° C. After several lots of material accumulated, the material may be pooled and used to make other classes of products.

Thus, in one embodiment, the process takes material that is generated following perfusion decellularization or mammalian organs or portions thereof and optional gentle compression of organs. Material can be stored at 4° C. until material is to be dehydrated. For dehydration, in one embodiment, material may be sized into approximately 1 “×1” pieces and then placed in a laminar flow hood (LFH) in a sterile environment. Pieces may be left for period of about 24 to 48 hours on an engaged LFH, or until they appear fully dehydrated. Complete dehydration may be determined by a visually distinct color change in the material. After drying and processing, material may be analyzed for moisture content.

Following dehydration, the dried decellularized ECM may then be milled, e.g., cryomilled, and optionally sized, to produce a fine ECM powder (having particles) that can be packaged, used aseptically or sterilized via various methods included e-beam or gamma radiation. The powder ECM can then be further formulated through enzymatic digestion to provide for an increased surface area that enables a printable ink for 3D printing.

### Extrusion-Based 3D Printing Methods

The particles produced by dehydration and sizing may be employed in extrusion-based 3D printing methods, such as fused deposition modeling (FDM) and direct ink writing (DIW), to fabricate devices and scaffolds for tissue engineering applications. In these methods an ink is forced through a nozzle as a viscous liquid following a predefined path determined by a computer model to build up a 3D object layer-by-layer. Solvent-cast direct-writing inks include hydrogels that maintain structure following extrusion.

### Inkjet Printing

Inkjet printing enables disposition of very small volumes (1-100 picoliters) of individual droplets from, a nozzle to a printing surface with the goal of forming structures post-solidification. Multinozzle inkjet print heads containing several hundred individual nozzles have been developed to accelerate the printing process. Inkjet printers are classified into two groups based on the droplet generation mechanism: continuous inkjet (CIJ) printing and drop-on-demand (DOD) inkjet printing. In CIJ printing continuous stream of drops (around 100 μm in diameter) are produced and unused ink is recycled. In DOD inkjet printing, individual drops (in the range of 25 to 50 μm in diameter) are generated when required. DOD type printers are commonly used for tissue engineering applications. The power of inkjet printing is the spatial resolution, e.g., placement of picoliter drops with ECM particles with positional accuracy (˜10 μm in x-y-axis). The most important properties of the ink are the viscosity and the surface tension. The viscosity of the ink should be suitably low usually below 10 cP (mPa s), under high shear rates, between 1×105 and 1×106−1. The surface tension determines the shape of the drop emerging from the nozzle and the shape of the drop on the substrate. Surface tension values of the inks generally range from 28 to 350 mN m−1. The resolution and accuracy of the printed object are determined by the interaction between adjacent drops (coalescence) and between individual drops and the substrate (such as surface tension and wettability). The liquid-to-solid phase transformation (i.e., solvent evaporation, temperature controlled transition, or gelling of a precursor solution) controls the final shape and size of the printed objected.

### Laser-Assisted 3D Bioprinting

Laser-assisted 3D bioprinting (LAB) is a non-contact, nozzle free printing process that directs laser pulses through a “ribbon” containing bioink, e.g., comprising the ECM particles described herein. The ribbon is supported by a titanium or gold layer capable of absorbing and subsequently transferring energy to the ribbon. The bioink and optionally cells are suspended on the bottom of the ribbon and when vaporized by the laser pulse, create a high-pressure bubble that eventually propels discrete droplets to the receiving substrate that lies just beyond the ribbon. This step is repeatedly performed to functionally create the 3D structures. LAB has demonstrated high retention of phenotype and cell viability after printing.

### Stereolithography

The stereolithography (SLA) method of bioprinting utilizes photopolymerization, a process in which a UV light or laser is directed in a pattern over a path of photopolymerizable liquid polymer, cross-linking the polymers into a hardened layer. As each layer is polymerized, the printing platform can be lowered further into the polymer solution allowing for multiple cycles to form a 3D structure.

### Hydrogel Inks

Hydrogels are three-dimensional polymer networks with the ability to hold a large quantity of water and provide microenvironments with tunable mechanics, degradation and functionalizability. Hydrogel inks may be referred to as bioinks when they contain cells and/or biochemical molecules such as ECM components. Inkjet, light-assisted, and extrusion-based 3D printing systems are the most common methods for hydrogel printing. The classical approach to designing a hydrogel ink is to formulate a polymer solution that forms a network immediately after printing. The network may be physically or chemically cross-linked in response to an external stimulus such as temperature, light, or ion concentration. The main advantage of the physically cross-linked hydrogels is the absence of chemical agents, which decreases the material toxicity. On the other hand, chemically cross-linked hydrogels are prepared through covalent bond formation and the resulting hydrogel is more resistant to mechanical forces hut it usually undergoes greater volume changes than physically cross-linked networks.

The physicochemical properties and gelation method of hydrogels formed with ECM can be tuned through chemical, physical and/or enzymatic mechanisms or modulated by Ihermai/pH sensitivity. ECM based bioinks may be prepared as a solution (e.g., 3%), e.g., and remain as a solution below 15° C., and gel at 37° C. within 30 min, which may be pH adjusted to physiological pH.

### EXEMPLARY EMBODIMENTS

In one embodiment, the disclosure provides a method of ECM particle formation. The method includes providing one or more portions of a decellularized mammalian organ comprising ECM, dehydrating the one or more portions at ambient temperature (e.g., in the absence of added heat) and optionally under a positive air flow; and subjecting the dehydrated portions to milling, thereby providing a population of particles of ECM. In one embodiment, the portions are compressed prior to dehydration. In one embodiment, the ECM is liver ECM. In one embodiment, the liver ECM is porcine or human liver ECM. In one embodiment, the portions are dehydrated at a temperature from about 1° C. to about 30° C. In one embodiment, tire portions are dehydrated at a temperature from about 4° C. to about 25° C., In one embodiment, the portions are dehydrated at a temperature from about 15° C. to about 25° C. In one embodiment, the portions are dehydrated at a temperature from about 20° C. to about 25° C. In one embodiment, the portions are dehydrated at a temperature from about 10° C. to about 20° C. In one embodiment, the portions are in a biologically compatible solution prior to dehydration, e.g., the solution comprises water or PBS. In one embodiment, the milling produces a population where at least 90% of the particles are less than about 2 mm in size. In one embodiment, the milling produces a population where at least 90% of the particles are less than about 0.15 mm in size. In one embodiment, the method further comprises separating the population of particles by size. In one embodiment, the separation is conducted by sieving. In one embodiment, the method further comprises subjecting the particles to enzymatic digestion. In one embodiment, the enzymatically digested particles comprise high molecular weight collagen, e.g., at least 150 kDa. In one embodiment, the population has a moisture content of less than about 10%, 5%, 2% or 1%.

The population of particles prepared by the method may be used alone or to form a mixture, e.g., a gel such as a hydrogel or bioink. The gel or ink may be used to form structures using 3D printing processes.

The invention will be further described by the following non-limiting examples.

### Example 1

Dried and blended perfusion decellularized liver samples were sized in a laminar flow hood. Different size pieces, e.g., 0.5 inch by 0.5 inch or 1 inch by 1 inch pieces (about 15 grams), of decellularized liver ECM (MM or MD) were loaded into a polycarbonate grinding vial. The content of the vials was subjected to grinding in a SPEX Cryogrinder. Two different protocols were used. One included 4 grinding phases, 10 CPS, 5 minute precool, 2 minute cool. The other protocol included 2 grinding phases, 10 CPS, 5 minute precool, 2 minute cool. The products of both were sifted in a 2 mm sieve for particle size analysis. The product of 4 grinding phases had 144 mg>2.0 mm (1.1%) and 13,051 mg<2.0 mm (98.9%). The product of 2 grinding phases had 233 mg>2.0 mm (2.2%) and 10,228 mg<2.0 mm (97.8%). Each product (approximately 1500 mg) was packaged in non sterile 5 mL amber glass vials and sealed with a butyl stopper and aluminum seal. Moisture content analysis was conducted (70° C.). Table 1 shows the data for two different samples for each protocol and original source (MM or MD, both liver derived perfusion decellularized ECM).

TABLE 1

| | Test #1- | Test#2- |
|---|---|---|
| Sample | Moisture content | Moisture content |
| MM (1 × 1), 4 phases | 7.19%, 765 mg | 7.00%, 645 mg |
| MM (1 × 1), 4 phases | 6.14%, 650 mg | 6.89%, 754 mg |
| MM (0.5 × 0.5), 4 phases | 6.35%, 727 mg | 6.26%, 847 mg |
| MM (1 × 1), 2 phases | 5.76%, 712 mg | 5.61%, 767 mg |
| MD (varied sizes), 4 phases | 7.80%, 718 mg | 7.17%, 849 mg |
| MD (varied sizes), 2 phases | 5.83%, 857 mg | 6.30%, 668 mg |
| MD (varied sizes), 4 phases | 6.91%, 694 mg | 7.64%, 982 mg |

The data in Table 1 shows that dehydration in a laminar flow hood at ambient temperature results in <10% moisture content.

### Example 2

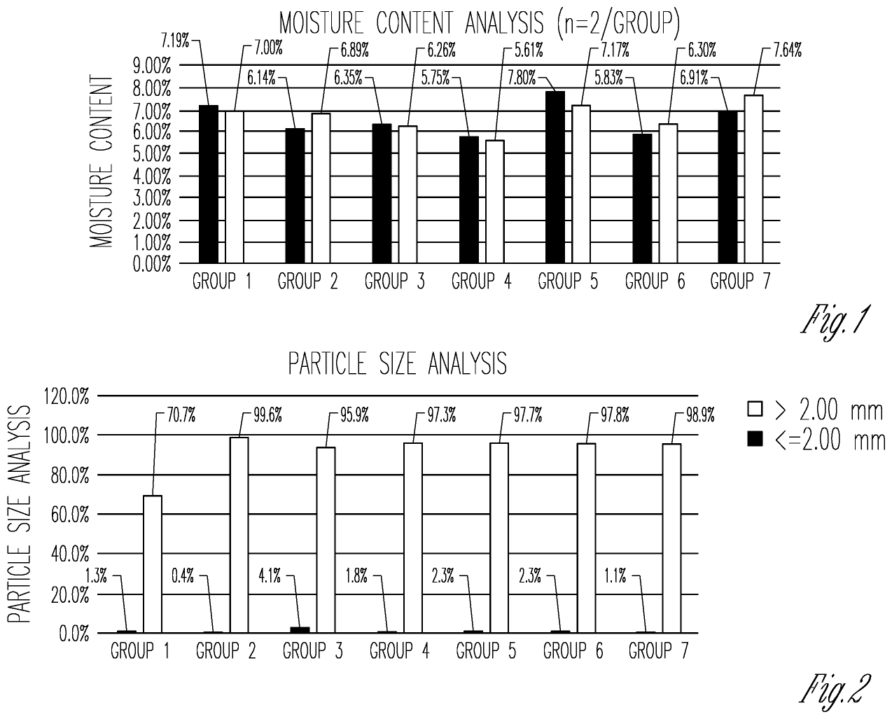

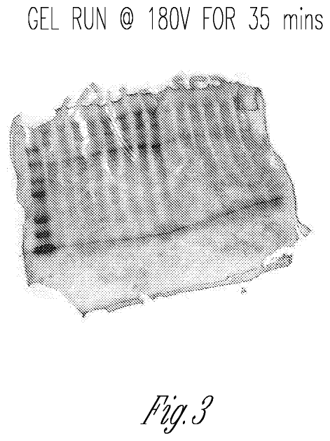

The data in FIG. 1 shows that compression and dehydration of the 7 sample types in Example 1 do not eliminate high molecular weight collagen (e.g., collagen>150 kDa) from ECM. FIG. 2 has particle size analysis for the 7 sample types in Example 1.

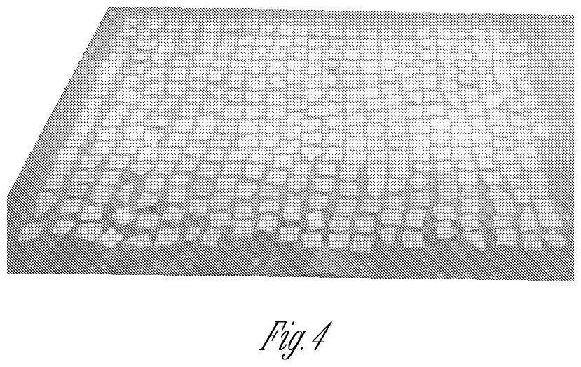

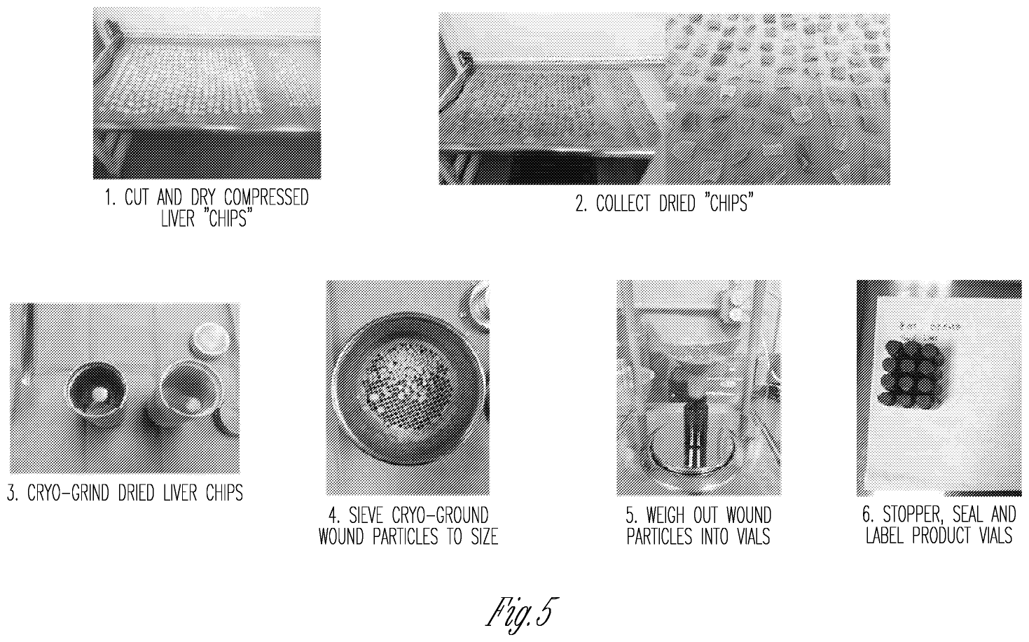

To determine how enzyme digestion alters structure, 26.2 mg pepsin (Sigma), 26.2, n×L H₂O and 262 microliters 1 N HCl were mixed. 500 microliter reactions of 1 mg/mL pepsin in 10 mM HQ with 1, 2.5, 5, and 10 mg of cryomilled decellularized, dehydrated ECM particles were split into 2 tubes and incubated at room, temperature or 4° C. for about 20 minutes, after which IN NaOH (55 microliters) was added to half of the samples to stop the reaction. Samples were diluted 1:1 in sample buffer and run on a 7.5% SDS-PAGE at 180 V for 35 minutes (FIG. 3).

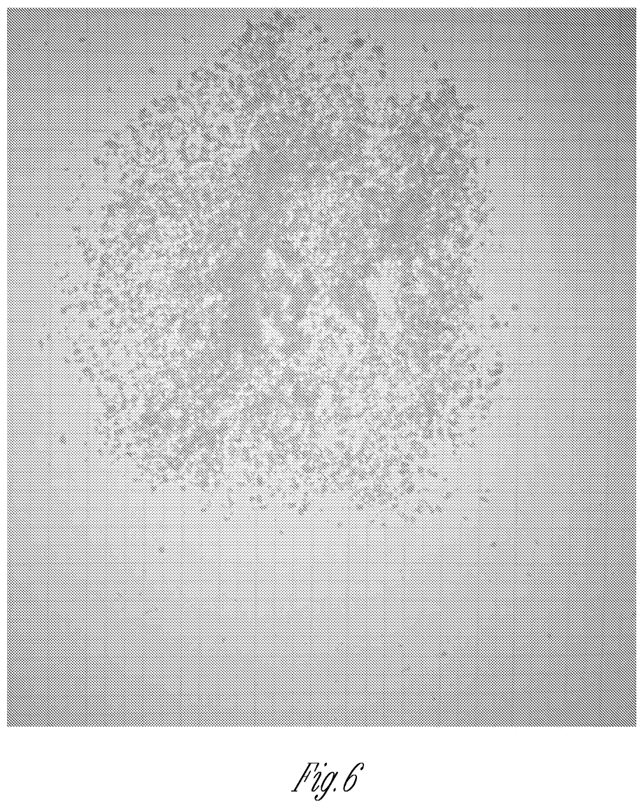

FIG. 5 provides an overview of steps in one embodiment of the method.

### Example 3

### Test Conditions:

6 vials of cryomilled liver particles were packaged and split into 3 groups (Table 2 summarizes Example 1)
   Control: Baseline MC %
   Positive Control: Vials were opened over 72 hours incubation in humid environment at 37° C.
   Test: Vials remained sealed over 72 hours incubation in humid environment at 37° C.

| Condition | Negative Control | Positive Control | Test |
|---|---|---|---|
| Moisture content | 5.11% | 14.55% | 5.61% |
| Moisture content | 6.21% | 13.92% | 5.97% |

TABLE 2

| | | | | | Moisture Content analysis | | | | Particle size | |
|---|---|---|---|---|---|---|---|---|---|---|
| | Milling | | | | Test 1 | | Test 2 | | Analysis | |
| | | | Vial Fill | # of | load | | load | | % > | % < |
| Group | Material | Size | (g) | Phases | (mg) | MC % | (mg) | MC % | 2.00 mm | 2.00 mm |
| 1 | dECM- | 1 × 1 | 8.94 | 4 | 965 | 7.19 | 645 | 7.00 | 1.30 | 70.70 |
| | Liver | | | | | | | | | |
| 2 | dECM- | 1 × 1 | 11.20 | 4 | 650 | 6.14 | 754 | 6.89 | 0.40 | 99.60 |
| | Liver | | | | | | | | | |
| 3 | dECM- | 0.5 × 0.5 | “approx. | 4 | 727 | 6.35 | 847 | 6.26 | 4.10 | 95.90 |
| | Liver | | ⅓ full” | | | | | | | |
| 4 | dECM- | 1 × 1 | 11.26 | 2 | 712 | 5.76 | 767 | 5.61 | 1.80 | 97.30 |
| | Liver | | | | | | | | | |
| 5 | dECM- | Varied | 12.40 | 4 | 718 | 7.80 | 849 | 7.17 | 2.30 | 97.70 |
| | Liver | sizes | | | | | | | | |
| 6 | dECM- | Varied | 15.00 | 2 | 857 | 5.83 | 668 | 6.30 | 2.30 | 97.80 |
| | Liver | sizes | | | | | | | | |
| 7 | dECM- | Varied | 15.00 | 4 | 694 | 6.91 | 982 | 7.64 | 1.10 | 98.90 |
| | Liver | sizes | | | | | | | | |

### Example 4

In one embodiment, one or more portions of dehydrated perfusion decellularized mammalian organ comprising extracellular matrix (ECM) are subjected to milling, thereby providing a population of particles of ECM. In one embodiment, the portions are compressed prior to dehydration. In one embodiment, the ECM is liver ECM, e.g., porcine or human liver ECM. In one embodiment, the portions are dehydrated at a temperature from about 1° C. to about 30° C. In one embodiment, the portions are in a physiologically compatible solution prior to dehydration, e.g., the solution comprises water or PBS. In one embodiment, the milling produces a population where at least 90% of the particles are less than about 2 mm in size find optionally greater than about 0.01 mm in size. In one embodiment, the milling produces a population where at least 90% of the particles are less than about 0.15 mm in size and optionally greater than about 0.01 mm in size. In one embodiment, the method further comprises separating the population of particles by size. In one embodiment, the separation is conducted by sieving, subjecting the population to acoustic energy, subjecting the population to density separation or by applying a fluid. In one embodiment, the method further comprises subjecting the particles to enzymatic digestion. In one embodiment, the enzymatically digested particles comprise high molecular weight collagen. In one embodiment, the population has a moisture content of less than about 10%.

In one embodiment, prior to dehydration the extracellular matrix is inflated with a gas. In one embodiment, the extracellular matrix after inflation has a height that is greater than about 0.2 cm up to about 0.6 cm, e.g., about 0.3 cm to about 0.5 cm, including about 0.4 cm, relative to that of a corresponding uninflated extracellular matrix. For example, the extracellular matrix of a liver lobe of a large mammal after inflation has a height that is greater than about 0.3 cm to about 0.5 cm, including about 0.4 cm, relative to that a corresponding uninflated extracellular matrix of a liver lobe. In one embodiment, the extracellular matrix after inflation has a height that is increased by at least 25% up to 1000%, e.g., at least 50% up to about 500% or about 75% up to about 250%, relative to a corresponding uninflated extracellular matrix. In one embodiment, the gas filled decellularized extracellular matrix of an organ or tissue has a shape, size or volume that is about 25% to 125%, for example, about 50% to about 150% or about 75% to about 110%, feat of the corresponding native organ or tissue. The active introduction of a gas, for instance a vapor (a gas having particles or droplets), into a decellularized extracellular matrix of an organ or tissue via its natural vasculature or any other conduit, e.g., duct or cavity, provides for a gas filled decellularized extracellular matrix of an organ or tissue matrix that is expanded relative to its non-inflated shape, and in some embodiments has the original shape of the native (cellularized) organ or tissue prior to decellularization. The shape of the gas filled decellularized extracellular matrix, for example one filled with air, is retained as a result of the gas being trapped within the tissue or organ and filling the spaces originally occupied by ceils. In one embodiment, the gas comprises normal air, CO₂, argon, nitrogen, or oxygen, or any combination thereof.

In one embodiment, a dehydrated planar configuration of a perfusion decellularized mammalian organ comprising ECM, or one or more portions thereof, is subjected to cryomilling, thereby providing a population of particles of ECM. In one embodiment, fee planar configuration or the one or more portions are compressed. In one embodiment, the ECM is liver ECM. In one embodiment, the planar configuration or the one or more portions are dehydrated at a temperature from about 1° C. to about 30° C. In one embodiment, the milling produces a population where at least 90% of the particles are less than about 2 mm in size. In one embodiment, the milling produces a population where at least 90% of the particles are less than about 0.15 mm in size. In one embodiment, the method further comprises separating the population of particles by size. In one embodiment, the method further comprises subjecting the particles to enzymatic digestion. In one embodiment, the population has a moisture content of less than about 10%.

A population of particles produced by the method, e.g., wherein at least 95% of the particles are less than about 2 mm in size or are less than about 0.15 mm in size, or a gel comprising fee population, may be employed in 3D printing or in wound therapy. In one embodiment, the population or gel is used for a biologic ink for 3D printing. In one embodiment, fee 3D printed structure is used for functional testing of embedded cells, e.g., the cells may be added after the 3D printed structure is formed. In one embodiment, the population or gel is infused or added to or mixed with a different bioink to create a suspended ECM particle composite ink. For example, the different bioink may include cells, proteins such as one or more growth factors or other scaffold materials, e.g., agarose, gelatin, Pluronics (poloxamers), alginate, collagen, hyaluronic acid, fibrin, silk, or cellulose.

In one embodiment, the dehydration and cryomilling of perfusion decellularized organ portions results in particles with a moisture content of about 5-20% depending on the environmental conditions.

In one embodiment, the overall size of cryomilled particles range from 0.02 mm to about 4 mm depending on the conditions used during cryomilling.

In one embodiment, cryomilled organ particles are added to 3D printing ink for the printing of 3D constructs containing whole organ derived ECM.

In one embodiment, cryomilled organ particles are digested with various enzyme(s) to make a solution capable of being utilized as ink in 3D printing. In one embodiment, enzymatic digestion results in ECM sizes of less than 2 mm, e.g., less than 1.8, 1.6, 1.4, 1.2, or 1.0 mm.

In one embodiment, the 3D printed structures based off of whole organ portions may be implanted into a mammal.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of ECM particle formation, comprising:
providing one or more portions of perfusion decellularized mammalian organ comprising extracellular matrix (ECM);
dehydrating the one or more ECM portions at an ambient temperature; and
subjecting the dehydrated ECM portions to milling, thereby providing a population of particles of ECM.

2. The method of claim 1 wherein the portions are compressed prior to dehydration.

3. The method of claim 1 wherein prior to dehydration, the one or more portions are inflated with a gas.

4. The method of claim 3 wherein the perfusion decellularized mammalian organ comprising extracellular matrix is inflated with a gas prior to providing the one or more portions thereof.

5. The method of claim 1 wherein the ECM is liver, heart, lung or kidney ECM.

6. The method of claim 3 wherein the ECM is porcine or human.

7. The method of claim 1 wherein the portions are dehydrated at a temperature from about 1° C. to about 30° C.

8. The method of claim 1 wherein the portions are in a physiologically compatible solution prior to dehydration.

9. The method of claim 8 wherein the solution comprises water or PBS.

10. The method of claim 1 any one of claims 1 to 9 wherein the milling produces a population where at least 90% of the particles are less than about 2 mm in size, or the milling produces a population where at least 90% of the particles are less than about 0.15 mm in size.

11. (canceled)

12. The method of claim 1 further comprising separating the population of particles by size.

13. The method of claim 12 wherein the separation is conducted by sieving,
subjecting the population to acoustic energy, subjecting the population to density separation or using fluid.

14. The method of claim 1 further comprising subjecting the particles to enzymatic digestion.

15. (canceled)

16. The method of claim 1 wherein the population has a moisture content of less than about 1.0%.

17. The method of claim 1 wherein
a planar configuration of the one or more portions of the perfusion decellularized mammalian organ comprising ECM is dehydrated and optionally subjected to cryomilling, thereby providing a population of particles of ECM.

18-25. (canceled)

26. A population of particles produced by the method of claim 1, wherein optionally at least 95% of the particles are less than about 2 mm or 0.15 mm in size.

27-28. (canceled)

29. A gel comprising the population of claim 26 which optionally comprises mammalian cells.

30-34. (canceled)

35. A method of using a bioink for 3D printing, comprising:
Providing a bioink composition comprising the population of claim 26; and
applying the bioink composition so as to form a 3D structure.

36. The method of claim 35 wherein the bioink composition further comprises cells.

37. The method of claim 35 wherein the bioink composition further comprises isolated protein or glycoproteins.

38-39. (canceled)
